(19) United States
(12) Patent Application Publication
(10) Pub. No.: US 2005/0033607 A1
(43) Pub. Date: Feb. 10, 2005

(54) Modeling reture on investment related to health care services

(76) Inventors: Archelle Georgiou, Wayzata, MN (US); William W. McGuire, Wayzata, MN (US)

Correspondence Address: Michael C. Barrett, Esq.;FULBRIGHT & JAWORSKI, L.L.P., Suite 2400, 600 Congress Avenue, Austin, TX, 78701, US

(21) Appl. No.: 10/788,674

(22) Filed: Feb. 27, 2004

Related U.S. Application Data
(60) Provisional application No. 60/450,440, filed on Feb. 27, 2003.

(51) Int. Cl. G06F 17/60; A61B 13/00

(52) U.S. Cl. 705/2; 705/35

(57) ABSTRACT

Techniques for modeling savings associated with health care services. Savings and/or a return on investment is modeled using efficacy data, measured results, economic modeling methodologies, and published data.

This application claims priority to, and incorporates by reference, U.S. Provisional Patent Application Ser. No. 60/450,440, which was filed Feb. 27, 2003.

### BACKGROUND OF THE INVENTION

1. Field of the Invention

The present invention generally relates to health care services. More particularly, the invention relates to modeling the return on investment associated with aspects of managed care.

2. Background

The accurate modeling of return on investment is important in the arena of health care services. Before implementing any new procedures, it is important to estimate the degree to which those new procedures will save money. This is especially important if the implementation of those new procedures is, in itself, costly.

Although they exhibit some usefulness, conventional models for estimating a return on investment (ROI) suffer from significant drawbacks. Namely, conventional models do not take into account or utilize sets of data particularly important for health care services. Consequently, conventional models fail to accurately estimate the ROI for aspects of managed care.

Thus, a significant need exists for the techniques described and claimed in this disclosure. In particular, what is needed is an improved model for ROI that can be readily applied to the field of health care. Such a model would allow practitioners to reliably predict the ROI in a wide range of situations, utilizing a more-complete set of the most pertinent data.

### SUMMARY OF THE INVENTION

Particular shortcomings of the prior art are reduced or eliminated by the techniques discussed in this disclosure.

In one respect, the invention involves a method in which savings associated with health care services are modeled using a) efficacy data, b) measured results, c) economic modeling methodologies, and d) published data.

In another respect, the invention involves a method for modeling savings associated with health care services. A savings is determined based upon efficacy data from published research. A savings based upon measured results is determined, the measured results including one or more of the following savings components: gap closure savings, non-coverage determinations, cost avoidance, and productivity/revenue. A savings based upon economic modeling methodologies is determined, the economic modeling methodologies including savings assumptions. Finally, a savings based upon published data is determined, the published data including one or more of the following components: clinical trials or observational data concerning complications or adverse events, published cost and savings estimates, and published wage data.

In another respect, the invention involves a method for modeling savings associated with health care services. A savings arising from the closure of gaps in health care is determined. A savings arising from a prospective review for non-covered health services is determined. A savings arising from health-related reminder programs is determined. Finally, a savings arising from decreased absenteeism is determined.

As used herein, “a” and “an” shall not be strictly interpreted as meaning “one” unless the context of the invention necessarily and absolutely requires such interpretation.

Other features and associated advantages will become apparent with reference to the following detailed description of specific embodiments in connection with the accompanying drawings.

### BRIEF DESCRIPTION OF THE DRAWINGS

The techniques of this disclosure may be better understood by reference to one or more of these drawings in combination with the detailed description of illustrative embodiments presented herein. Identical or similar elements may use the same element number. The drawings are not necessarily drawn to scale.

FIG. 1 is a schematic diagram displaying how a rigorous and valid savings estimation may be built, according to embodiments of this disclosure, by considering data, results, and methodologies from the different illustrated areas.

FIG. 2 is a schematic diagram providing details of data, results, and methodologies for a saving estimation, according to embodiments of the disclosure.

FIG. 3 is a schematic diagram displaying different savings components taken into account by different embodiments of ROI modeling tools of the present disclosure.

FIG. 4 is a monthly estimate saving chart based on a set of assumptions, according to embodiments of the disclosure.

FIG. 5 is a per member per month return on investment chart, according to embodiments of the disclosure.

FIG. 6 is a summary chart of a short-term saving component, according to embodiments of the disclosure.

FIG. 7 is a summary chart displaying the cost of treatment for a type of medical condition, according to embodiments of the disclosure.

FIG. 8 is a summary chart displaying the cost of treatment for a type of medical condition, according to embodiments of the disclosure.

FIG. 9 is a summary chart of data sources providing cost estimates for certain medical conditions, according to embodiments of the disclosure.

FIG. 10 is a summary chart displaying non-covered components cost, according to embodiments of the disclosure.

FIG. 11 is a summary chart of data sources providing cost analysis, according to embodiments of the disclosure.

FIG. 12 is a summary chart of productivity and revenue data elements, according to embodiments of the disclosure.

FIG. 13 is a summary chart displaying a productivity and revenue program, according to embodiments of the disclosure.

FIG. 14 is a summary chart displaying the savings yielded from a reminder program, according to embodiments of the disclosure.

FIG. 15 is a summary chart displaying a reminder program for a pre-screening test, according to embodiments of the disclosure.

### DESCRIPTION OF ILLUSTRATIVE EMBODIMENTS

Techniques of this disclosure aim to address or eliminate shortcomings in the prior art by utilizing a more-complete set of the most pertinent data for modeling the ROI associated with aspects of managed care.

Advantages of the present invention include the fact that embodiments of the savings models are transparent. There are no “black box” assumptions or data points. The models may be set up to be interactive, while allowing for multiple modeling scenarios. The models are also flexible. Savings analysis may be modeled to reflect unique benefit plan design features and/or customer priorities. The models may also be continually updated to reflect new data, research, and evidence. Accordingly, the user may receive results from this model that are customized to their needs.

In one embodiment, a ROI may be modeled to determine the savings to a user who implements health care programs such as those disclosed in pending U.S. patent application Ser. No. 09/837,724 entitled: “Health Care Management System and Method,” which is incorporated herein by reference in its entirety.

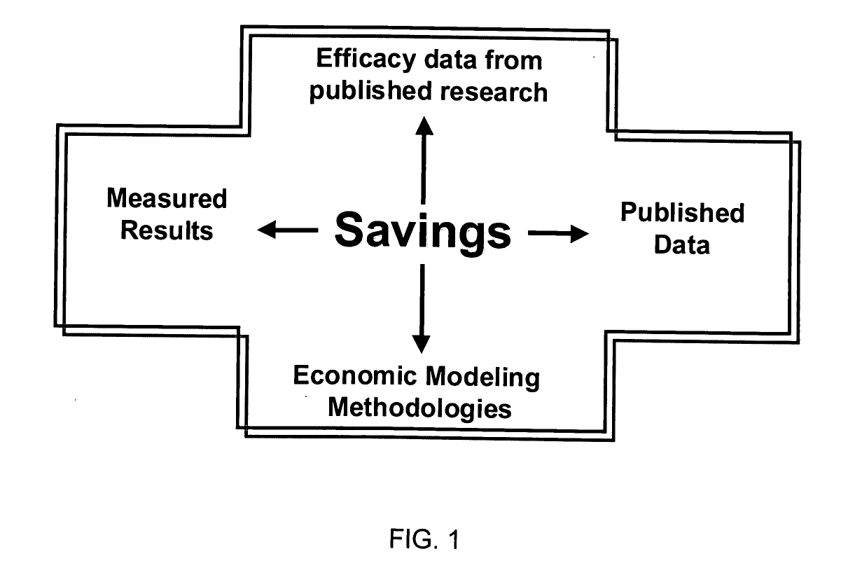

A rigorous and valid savings estimation may be built, according to embodiments of this disclosure, by considering data, results, and methodologies from the different illustrated areas. As illustrated in FIG. 1, a savings may be modeled by considering: a) efficacy data from published research, b) measured results, c) economic modeling methodologies, and/or d) published data. Using such data, results, and methodologies allows for the implementation of an ROI model that may be built on core analytical values and data and yields valid savings estimation.

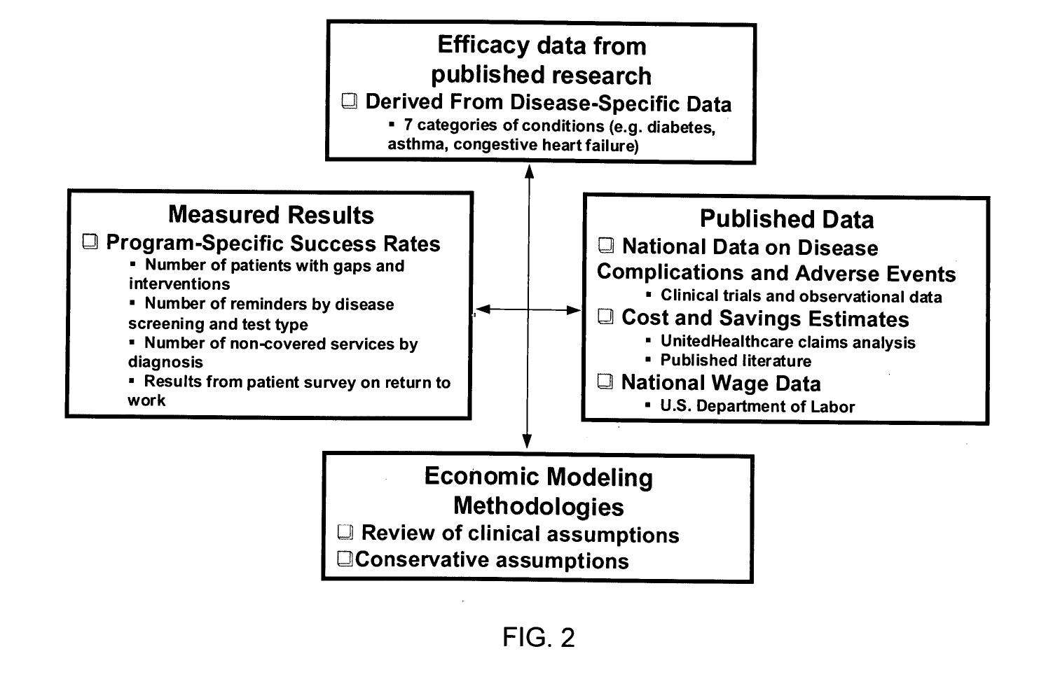

In particular, the data, results, and measurements may include a plurality of parameters, as illustrated in FIG. 2. In one embodiment, the efficacy data from published research may be derived from disease-specific data. For example, the efficacy data may be derived from one or more of the following: 1) asthma, 2) chronic renal failure, 3) congestive heart failure, 4) diabetes, 5) essential hypertension, 6) HIV/AIDS, and/or 7) other conditions.

The measured results may be based upon program-specific success rates. In particular, measured results may be based upon the following: 1) the number of patients with gaps in health care and interventions to fill such gaps, 2) the number of reminders by disease screening and test type, 3) the number of non-covered services by diagnosis, and/or 4) results from patient survey upon returning to work.

The economic modeling methodologies such as, but not limited to, 1) a review of clinical assumptions, and 2) conservative assumptions that may be used to aid in the estimation of savings.

The published data may be based on a variety of sources. For instance, it may be based on 1) national data on disease, 2) clinical trials and observational data concerning complications and adverse events, 3) cost and savings estimates from, for example, claims analysis and published literature, and/or 4) national wage data including but not limited to data from the U.S. Department of Labor.

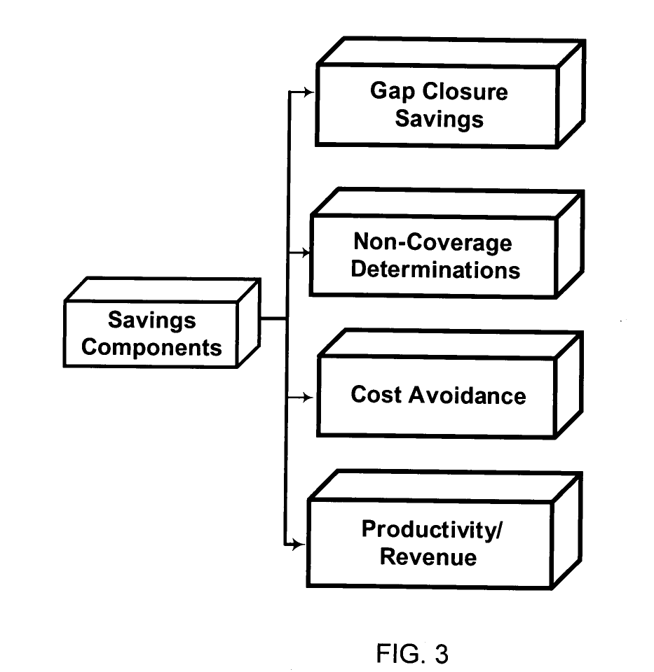

Referring to FIG. 3, a schematic diagram illustrates different savings components taken into account by different embodiments of ROI modeling tools of the present disclosure. As illustrated, savings components may include one or more of the following: 1) gap closure savings, 2) non-coverage determinations, 3) cost avoidance, and 4) productivity/revenue.

According to one embodiment, the gap closure savings relate to the financial impact of closing gaps in health care. Exemplary gaps may be: a lack of a specialist on a care team, a gap in filled prescriptions, and/or a gap created by poor diet compliance. As each of these gaps are completely or even partially closed, savings results. The non-coverage determinations relate to cost savings due to prospective review for non-covered services and application of evidence-based medicine. Cost avoidance relates to the modeled annual financial impact of reminder programs including prevention of adverse events and earlier detection of severe and costly diseases. Productivity/revenue relates to the financial impact of decreased absenteeism due to patients returning to work and normal activity as quickly as possible. Each of these components will be discussed in more detail below.

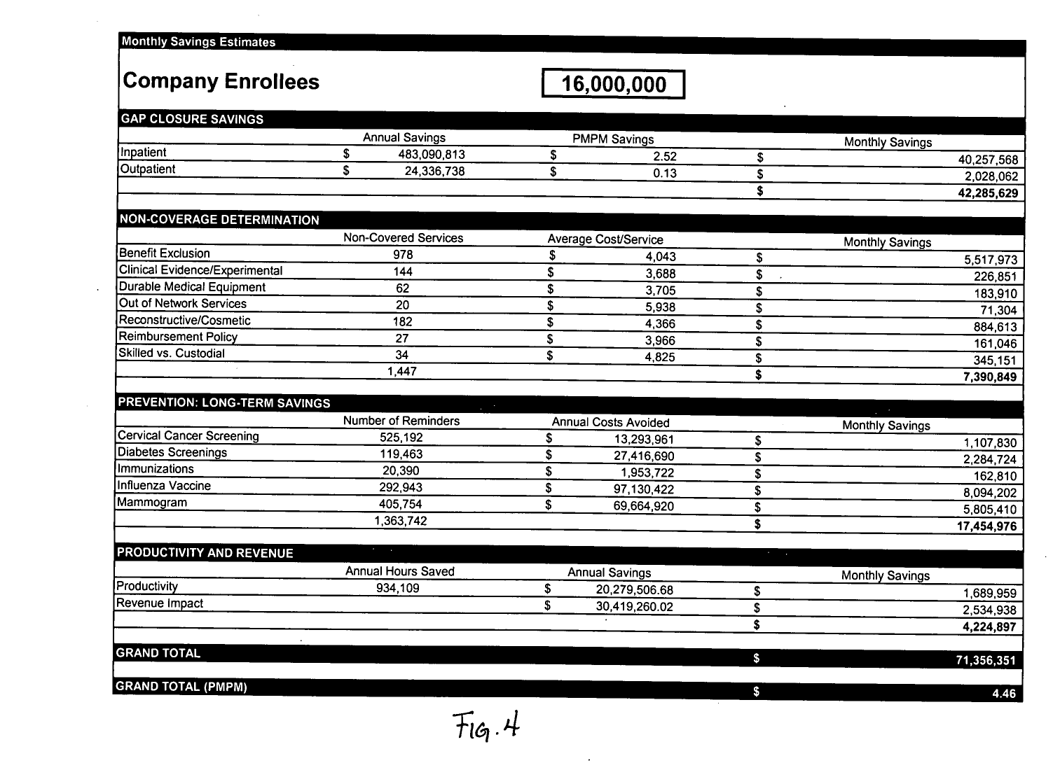

The cost-saving model may take into account a few assumptions that aids in the determination of the savings. For example, the model may be based on the assumption that the number of health plan enrollees served as a proportion to the overall health plan population and the distribution of health plan enrollees by disease area will be the same for any customer. Referring to FIG. 4, a monthly saving estimate model for 16 million enrollees may take into these assumptions to determine an effective health care management scheme while implementing cost-saving components.

Further, each cost saving component of the saving estimate model may take into account other assumptions to calculate the savings. For example, the Gap Closure Savings calculations may assume that there is a minimum impact of intervention on both inpatient and outpatient utilization. Additionally, the Non-Coverage Determination calculations may assume that the non-covered services, including but not limited to, out of network services, reconstructive or cosmetic surgeries, medical equipment, etc., would have been paid by the health plan if the non-coverage determination process was not in place. The Long-Term Prevention Savings, while focusing on long-term prevention and early detection of some diseases, may assume that a portion of the savings generated may be realized annually for certain criteria, such as but not limited to, early detection versus late detection and better disease management. The Productivity and Revenue assumes the time away from work, such as days lost due to illness, may generate costs associated with replacement workers and payment of wages and benefits for a period of non-production. The assumption may be the cost of replacement works and payment of wages for non-production may be equivalent to the wages paid to a full-time employee.

These cost saving components and the assumption of the model may generate a significant savings method. As illustrated in FIG. 4, the grand total savings of per member, per month (PMPM) multiplied by the number of enrollees in the management company yields a large saving for any given month.

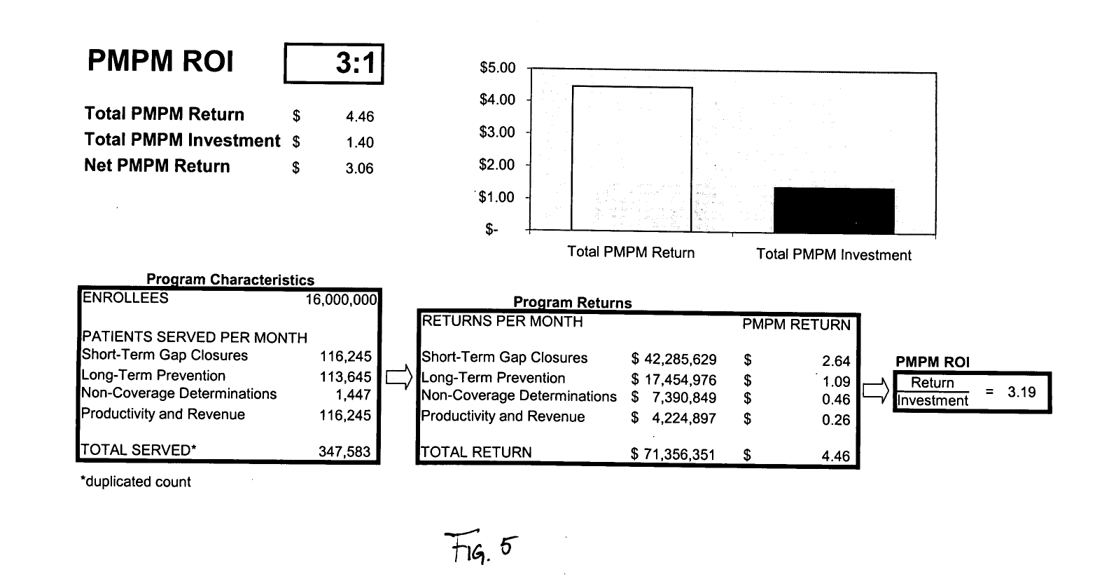

The PMPM savings may further be analyzed, as illustrated in FIG. 5. The total number of enrollees serve per month is a fraction of the total number of enrollees of the management company. As such, using the PMPM savings grand total from FIG. 4 divided by the total PMPM investment for the actual number of enrollees in a given month, the PMPM ROI is a 3:1 ratio.

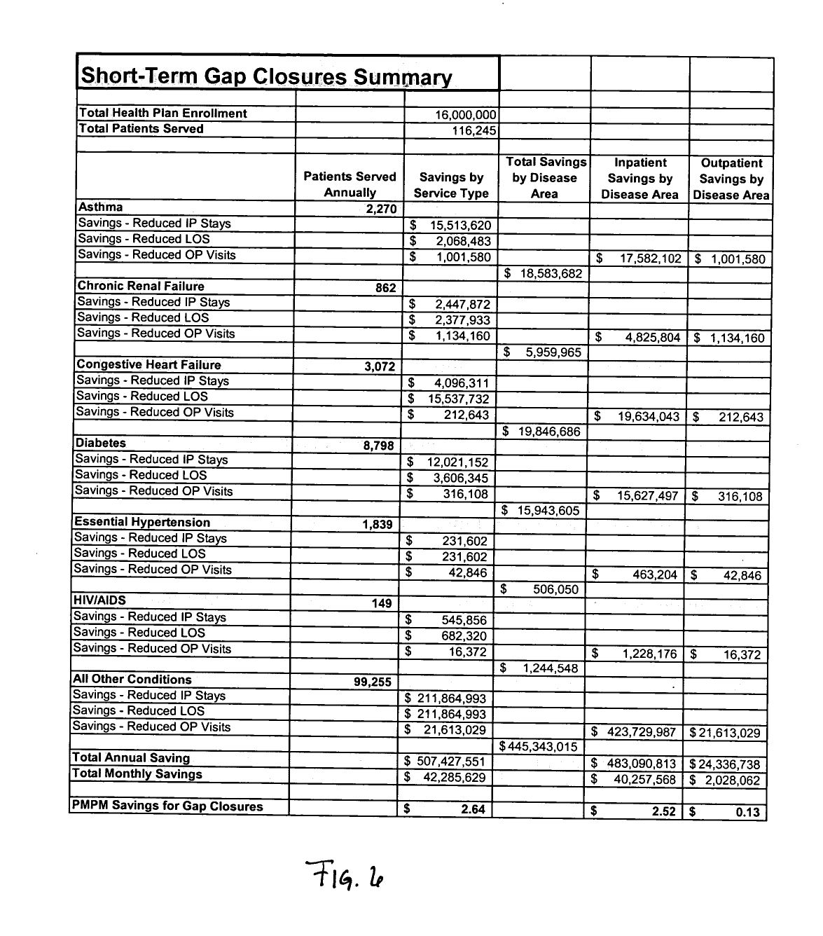

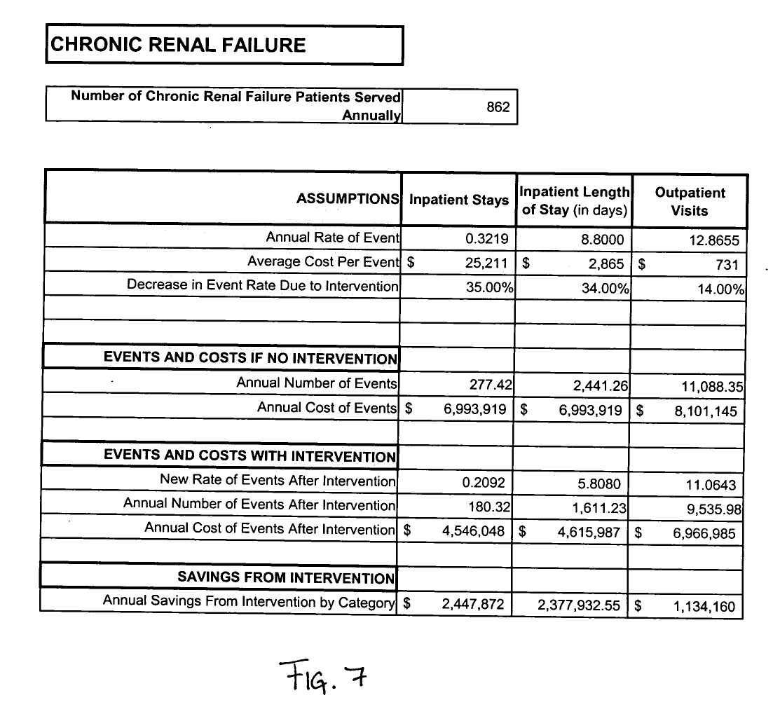

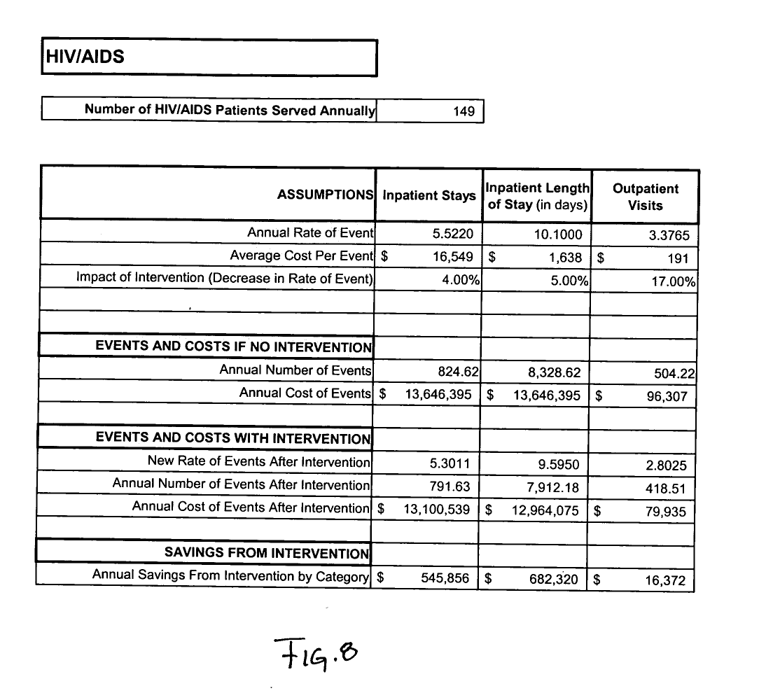

Each cost saving component may further be model based on efficacy data. For example, FIG. 6 illustrates a short-term gap closure summary on medical conditions, including, but not limited to, asthma, chronic renal failure, congestive heart failure, diabetes, essential hypertension, HIV/AIDs, and other conditions. For each medical condition outlined in FIG. 6, a summary chart may be provided to determine the impact of intervention as well as the overall savings by implementing an intervention program. Particularly, each medical condition may be generated from data sources that populate the cost per event field, as illustrated in FIG. 7 and FIG. 8. In particular, FIG. 7 is a summary of the costs of medical services provided to patients diagnosed with or at risk for chronic renal failure. Similarly, FIG. 8 is a summary chart for patients diagnosed with or at risk for contracting HIV/AIDS.

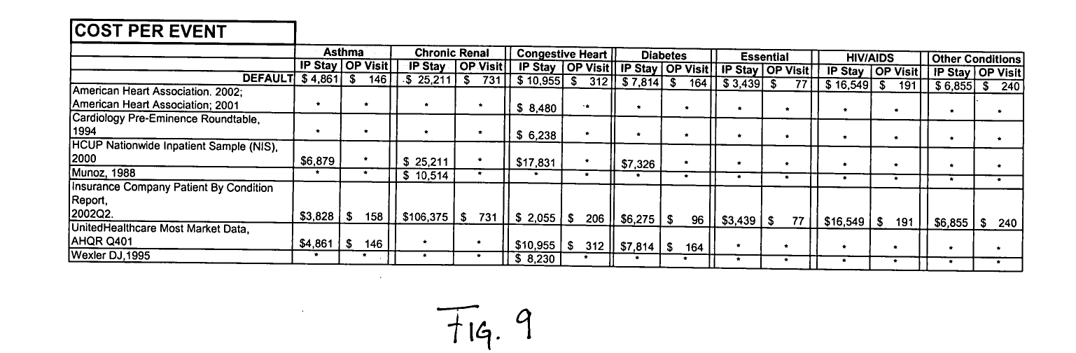

As mentioned above, the efficacy data may be provided by published sources that generate data showing the impact of an intervention field for each medical condition. For example, FIG. 9 illustrates a list of data sources that provide estimates for the cost of care for inpatients and outpatients for a given medical condition. These cost estimates may be able to determine the savings yielded when gap closure is implemented.

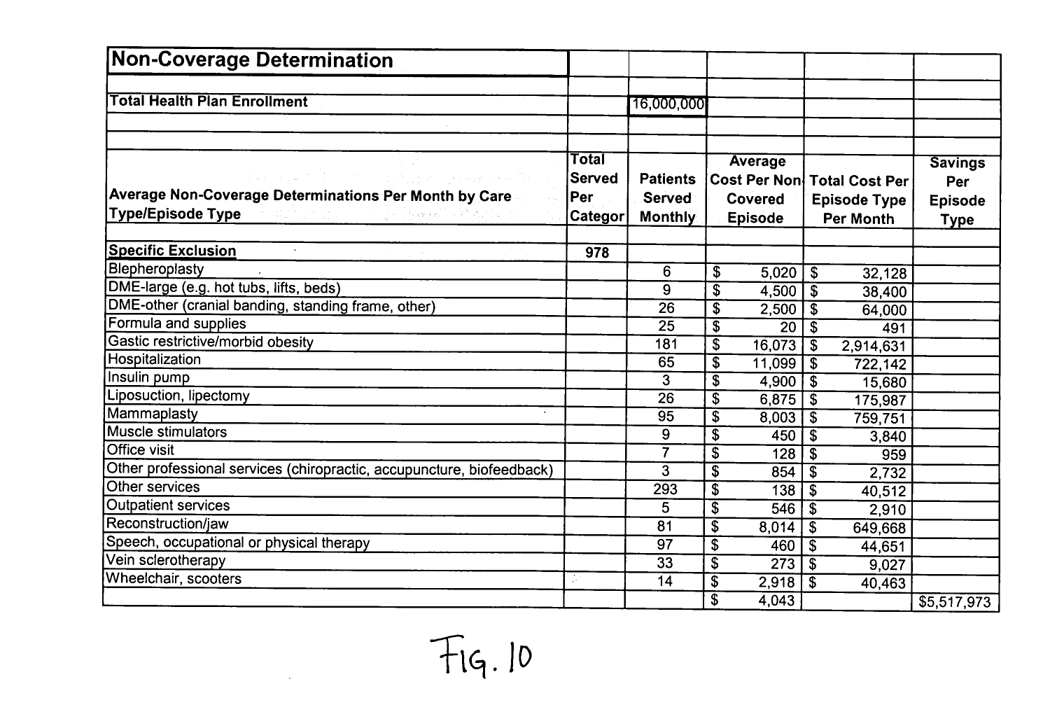

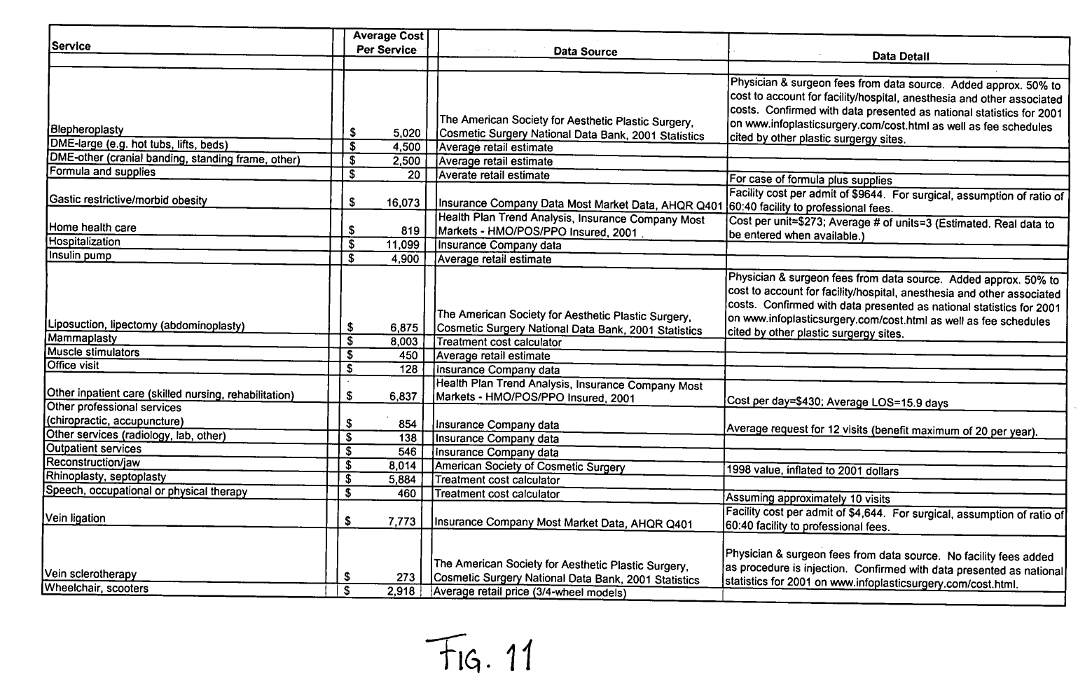

In another embodiment, a summary chart may be provided to determine what proportion of the total health plan population participates in non-coverage procedures e.g., medical procedures excluded from benefit plans, and the savings based on an episode type. Referring to FIG. 10, a summary of non-coverage procedures and the cost related to each procedure is displayed for an average number of patients that undergo these type procedures. It is noted the summary chart is not exhaustive of all types of non-coverage components. Particularly, other types of non-coverage components may include, but is not limited to, cosmetic procedures, medical equipment, and clinical evidence. The average cost of the listed procedures may be provided by data sources which have compiled the average cost, as illustrated in FIG. 11.

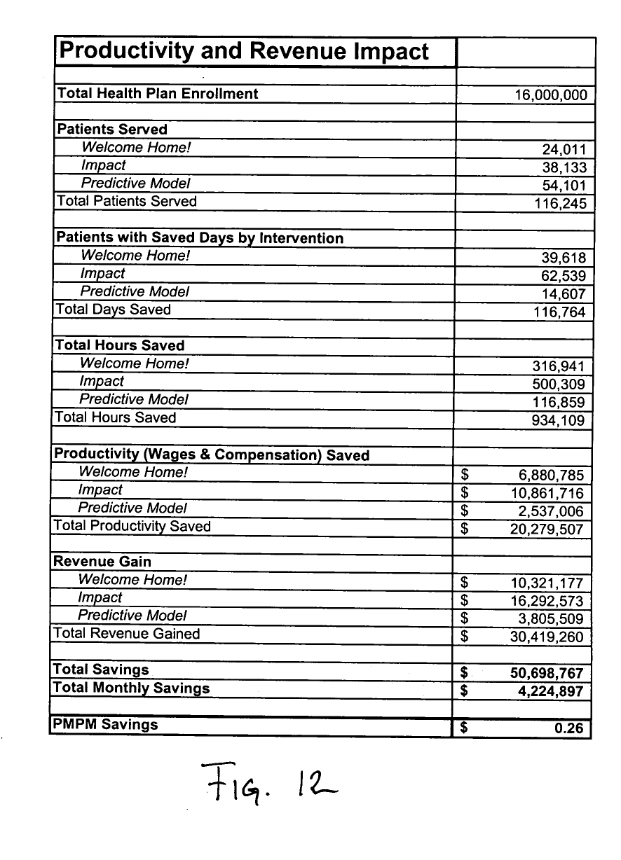

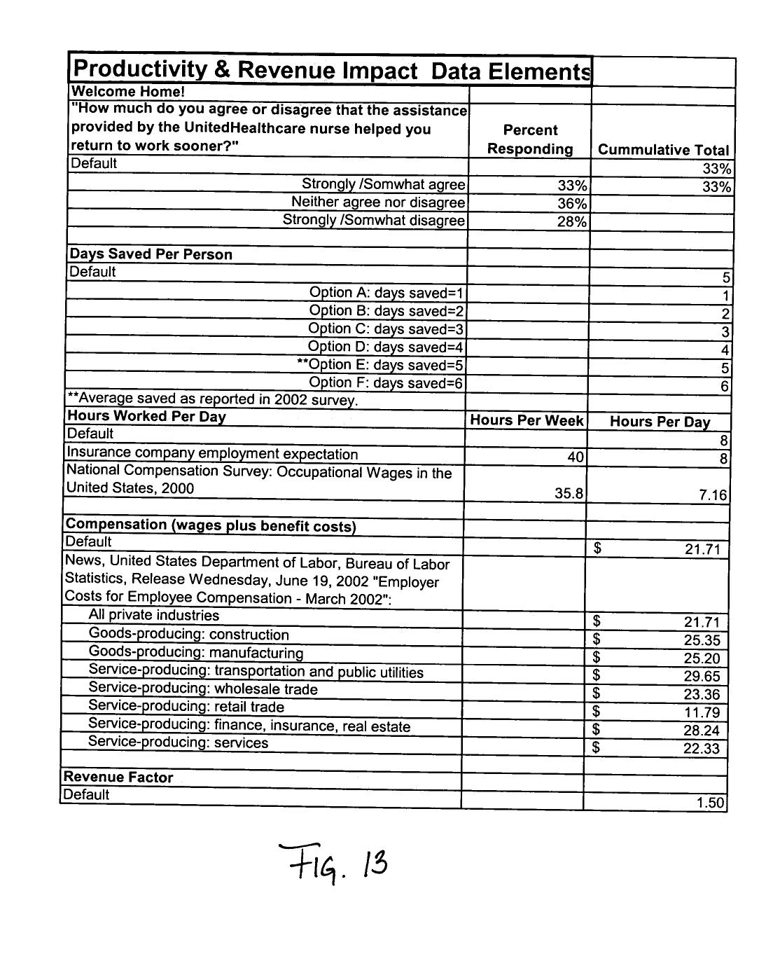

As mentioned above, the productivity and revenue assessments assumes the time away from work, such as days lost due to illness, may generate costs associated with replacement workers and payment of wages and benefits for a period of non-production. The assumption may be the cost of replacement works and payment of wages for non-production may be equivalent to the wages paid to a full-time employee. As such, the productivity and revenue impact may be analyzed to determine the savings associated. Referring to FIG. 12, a summary chart of the productivity and revenue impact is modeled. An intervention program may be implemented to save on long-term care and medical expenses for patients with risks to a certain medical condition. The intervention program reduces the number of hours and days spent on medical care, and thus generates a savings Referring to FIG. 13, an example of a data source from a program, particularly the Welcome Home program which provides data for populating the productivity and revenue impact model. Particularly FIG. 13 illustrates the most reliable or conservative value as the default value in determining the savings. It is noted that a similar summary chart may be provided for each program, e.g., the Impact program and the Predictive Model program, calculating the effects of productivity and revenue of employees.

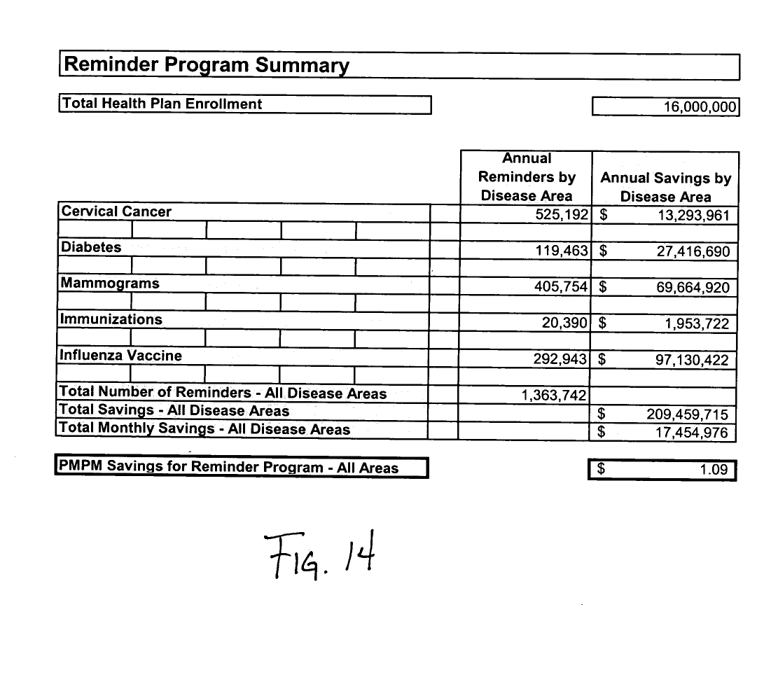

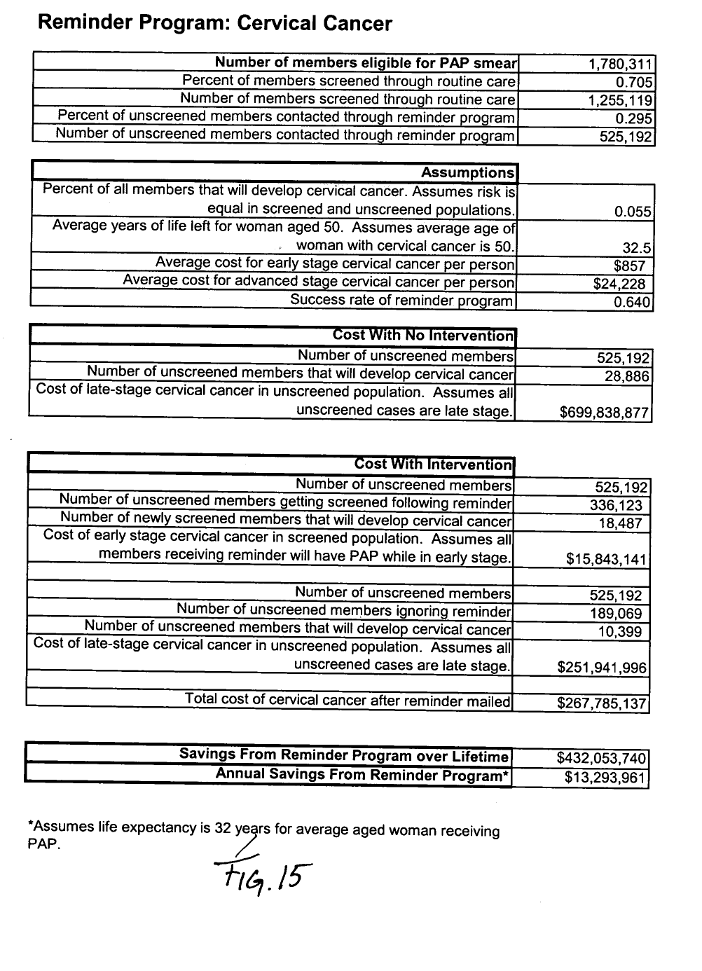

Preventative and pre-screening care may also contribute to the annual saving. Referring to FIG. 14, a summary chart is provided for a Reminder Program that notifies members of certain tests and vaccination, including, but not limited to cervical cancer screening, diabetes screening, mammograms, immunizations, and influenza vaccines. By implementing such a program, an annual savings may be realized. For example, the data for Reminder Program to screen for cervical cancer is illustrated in FIG. 15. Many assumptions may be made to calculate the cost of pap smears over the lifetime of woman with and without the Reminder program.

### EXAMPLES

The following examples are included to demonstrate specific, non-limiting embodiments of this disclosure. It should be appreciated by those of skill in the art that the techniques disclosed in the examples that follow represent techniques discovered by the inventors to function well in the practice of the invention, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the invention.

By implementing a plurality of summary charts, e.g., computer spreadsheets, a cost-saving model may be obtained. For example, data from published sources, measured results, economic considerations, may be saved into spreadsheets. A healthcare management system may access the spreadsheets and input data, such as the number of members enrolled in the healthcare plan, average patients seen monthly for a certain medical, conditions, etc. Varying assumptions such as a conservative or aggressive data may be change to reflect the model of interest. Example algorithms that calculate a ROI module using techniques described above are exhibited in Tabs A-J of co-pending U.S. Provisional Application 60/450,440, already incorporated in its entirety by reference.

These parameters may allow for accurate calculations prior to the implementation of a program. For example, a healthcare management company may want to initiate an intervention program. By using statistics from other healthcare services, data from published sources, the categorizing of medical conditions and data specific to the healthcare management system, ROI calculations may be easily obtained.

With the benefit of the present disclosure, those having ordinary skill in the art will comprehend that techniques claimed herein and described above may be modified and applied to a number of additional, different applications, achieving the same or a similar result. For example, one will recognize that the ROI tools disclosed herein may be applied to different fields other than health care services. The claims cover all modifications that fall within the scope and spirit of this disclosure.

### References

Each of the following is incorporated by reference in its entirety.
American Heart Association, 2002 Heart and Stroke Statistical Update. Dallas, Tex.: American Heart Association, 2001.
Bardenheier B, et al. Tetanus surveillance—United States, 1995-1997. Mor Mortal Wkly Rep CDC Surveill Summ, July 3;47(2):1-13, 1998.
Bruce D, Dickmeyer J. Don't overlook disease management programs for low-incidence, high-cost diseases to improve your bottom line. J Health Care Finance, 28(2):45-9, 2001.
Cardiology Pre-Eminence Roundtable. Beyond Four Walls: Cost Effective Management of Chronic Congestive Heart Failure. Washington, D.C.: The Advisory Board Company, 1994.
Caro J J, et al. Lifetime costs of complications resulting from type 2 diabetes in the U.S. Diabetes Care March, 25(3):476-81, 2002.
Carroll N V, et al. Economic burden of influenza-like illness in long-term care facilities. Am J Health Sys Pharm, June 15;58(12):1133-8, 2001.
Centers for Disease Control. Current trends impact of diabetes outpatient education program—Maine. Morbidity and Mortality Weekly, 31(23):307-8,313-4, 1982.
Centers for Disease Control and Prevention. Press Release: CDC releases first cervical cancer detection rates by race and ethnicity. http://www.cdc.gov/od/oc/media/pressre1/r010116a.htm.
Cox F M, et al. Cost of treating influenza in emergency department and hospital settings. Am J Manag Care, February;6(2):205-14, 2000.
Golaz A, et al. Epidemiology of diphtheria in South Dakota. SDJ Med, July;53(7):281-5, 2000.
Greineder D K, Loane K C, Parks P. Reduction in resource utilization by an asthma outreach program. Arch Pediatr Adolesc Med, 149(4):415-20, 1995.
Hay J W, Daum R S. Arch Fam Med, November-December;9(10):989-96, 2000.
Higgins J C, et al. Influence of an interventional program on resource use and cost in pediatric asthma. Am J Manag Care, 4(10):1465-9, 1998.
The HIV Research Network. Hospital and Outpatient Health Services Utilization Among HIV-Infected Patients in Care in 1999. Journal of Acquired Immune Deficiency Syndromes, 30:21-26, 2002.
Kasper E K, et al. A Randomized Trial of the Efficacy of Multidisciplinary Care in Heart Failure Outpatients at High Risk of Hospital Readmission. J Am Coll Cardiol, 39(3):471-80, 2002.
Knox D, Mischke L. Implementing a congestive heart failure disease management program to decrease length of stay and cost. J Cardiovasc Nurs, 14(1):55-74, 1999.
Krumholz H M, et al. Randomized trial of an education and support intervention to prevent readmission of patients with heart failure. J Am Coll Cardiol, 39(1):83-9, 2002.
Martin L M, et al. Cervical cancer incidence and screening: status report on women in the United States. Cancer Pract, May-June;4(3):130-4, 1996.
Mast E E, et al. Risk factors for measles in a previously vaccinated population and cost-effectiveness of revaccination strategies. JAMA, November 21;264(19):2529-33, 1990.
Meltzer M I, et al. The economic impact of pandemic influenza in the U.S. Priorities for intervention. Emerg Infect Dis, September-October;5(5):659-671, 1999.
Menzin J, et al. Potential short-term economic benefits of improved glycemic control: a managed care perspective. Diabetes Care, 24(1):51-5, 2001.
Merrill R M, Feuer E J. Risk-adjusted cancer-incidence rates (United States). Cancer Causes Control, September;7(5):544-52, 1996.
Morbidity & Mortality: 2002 Chart Book on Cardiovascular, Lung, and Blood Diseases. http://www.nhlbi.nih.gov/resources/docs/02_chtbk.pdt
Munoz E, et al. Diagnosis related groups, resource utilization, age, and outcome for hospitalized nephrology patients. Am J Kidney Dis, 11(6):481-8, 1988.
National Cancer Institute. Cervical Cancer: Backgrounder Facts and Figures. http://rex.nci.nih.gov/massmedia/backgrounders/cervical.html.
National Center for Health Statistics. Fast Stats: Influenza. http://www.cdc.gov/nchs/fastats/flu.htm.
National Center for Infectious Diseases. Influenza: The Disease. http://www.cdc.gov/ncidod/diseases/flu/fluinfo.htm.
Nichol K L, et al. Default derived from the following statistics. The efficacy and cost effectiveness of vaccination against influenza among elderly persons living in the community. N Engl J Med, September 22;331(12):778-84, 1994.
Nissenson A R, et al. Evaluation of disease-state management of dialysis patients. Am J Kidney Dis, 37(5):938-44, 2001.
Norlund A, et al. Cost of illness of adult diabetes mellitus underestimated if comorbidity is not considered. J Intern Med, July;250(1):57-65, 2001.
O'Brien J A, et al. Diabetes in Canada: direct medical costs of major macrovascular complications. Value Health, May-June;4(3):258-65, 2001.
O'Brien J A, et al. Diabetes in Canada: direct medical costs of major macrovascular complications. Value Health, May-June;4(3):258-65, 2001.
Orenstein W A, et al. The opportunity and obligation to eliminate rubella from the United States. JAMA, April 20;251(15):1988-94, 1994.
Paramore L C, et al. Impact of poorly controlled hypertension on healthcare resource utilization and cost. Am J Manag Care, 7(4):389-98, 2001.
Pradeep A, et al. Hospital utilization among chronic dialysis patients. J Am Soc Nephrol, 11(4):740-6, 2000.
Ramsey S, et al. Productivity and medical costs of diabetes in a large employer population. Diabetes Care, January;25(1):23-9, 2002.
Rauh R A, et al. A community hospital-based congestive heart failure program: impact on length of stay, admission and readmission rates, and cost. Am J Manag Care, 5(1):37-43 1999.
Riegel B, Carlson B, Kopp Z, LePetri B, Glaser D, Unger A. Effect of a standardized nurse case-management telephone intervention on resource use in patients with chronic heart failure. Archives of Internal Medicine, 162:705-12, 2002.
Rubin R J, Dietrich K A, Hawk A D. Clinical and economic impact of implementing a comprehensive diabetes management program in managed care. J Clin Endocrinol Metab, August;83(8)2635-42, 1998.
Schoell W M, et al. Epidemiology and biology of cervical cancer. Semin Surg Oncol, April-May;16(3):203-11, 1999.
Sosin D M, et al. Changing epidemiology of mumps and its impact on university campuses. Pediatrics, November;84(5):779-84, 1989.
Suh D C, et al. Impact of a targeted asthma intervention program on treatment costs in patients with asthma. Am J Manag Care, 7(9):897-906, 2001.
Van Loon F P, et al. Mumps surveillance—United States, 1988-1993. Mor Mortal Wkly Rep CDC Surveill Summ, August 11;44(3):1-14, 1995.
Vickery D M, et al. Effect of self-care education program on medical visits. JAMA, 250:2953-2956, 1983.
Wexler D J, et al. Predictors of costs of caring for elderly patients discharged with heart failure. Am Heart J, 142(2):350-7, 2001.
Wolstenholme J L, Whynes D K. Stage-specific treatment costs for cervical cancer in the United Kingdom. Eur J Cancer, November;34(12):1889-93, 1998.

1. A method comprising:
modeling savings associated with health care services using efficacy data, measured results, economic modeling methodologies, and published data.

2. A method for modeling savings associated with health care services, comprising:
determining a savings based upon efficacy data from published research;
determining a savings based upon measured results, the measured results comprising one or more of the following savings components: gap closure savings, non-coverage determinations, cost avoidance, and productivity/revenue;
determining a savings based upon economic modeling methodologies, the economic modeling methodologies comprising savings assumptions; and
determining a savings based upon published data, the published data comprising one or more of the following components: clinical trials or observational data concerning complications or adverse events, published cost and savings estimates, and published wage data.

3. The method of claim 2, further comprising calculating a return on investment (ROI) using one or more of the determined savings.

4. The method of claim 2, the efficacy data comprising data concerning one or more of the following conditions: asthma, chronic renal failure, congestive heart failure, diabetes, essential hypertension, and HIV/AIDS.

5. The method of claim 2, the savings assumptions comprising clinical assumptions or conservative assumptions.

6. Computer-readable medium comprising instructions for modeling savings associated with health care services using efficacy data, measured results, economic modeling methodologies, and published data.

7. The medium of claim 6, the instructions being embedded within a computer spreadsheet.

8. A method for modeling savings associated with health care services, comprising:
determining a savings arising from the closure of gaps in health care;
determining a savings arising from a prospective review for non-covered health services;
determining a savings arising from health-related reminder programs; and
determining a savings arising from decreased absenteeism.

9. The method of claim 8, further comprising calculating a return on investment (ROI) using one or more of the determined savings.

10. Computer-readable medium comprising instructions for determining a savings arising from the closure of gaps in health care;
determining a savings arising from a prospective review for non-covered health services;
determining a savings arising from health-related reminder programs; and
determining a savings arising from decreased absenteeism.

11. The medium of claim 10, the instructions being embedded within a computer spreadsheet.

12. The medium of claim 11, the instructions being embedded in different modules, each module corresponding to a different savings component.
